(19) United States
(12) Patent Application Publication
(10) Pub. No.: US 2010/0160713 A1
(43) Pub. Date: Jun. 24, 2010

(54) DEVICE AND METHOD FOR ELECTROMAGNETIC STIMULATION OF A PROCESS WITHIN LIVING ORGANISMS

(75) Inventor: Johannes Josephus Maria Cuppen, Veldhoven (NL)

Correspondence Address: WESTMAN CHAMPLIN & KELLY, P.A., SUITE 1400, 900 SECOND AVENUE SOUTH, MINNEAPOLIS, MN, 55402, US

(73) Assignee: QISC B.V., Veldhoven (NL)

(21) Appl. No.: 12/670,803

(22) PCT Filed: Jul. 18, 2008
(86) PCT No.: PCT/EP08/59482

§ 371 Date: Feb. 18, 2010

(30) Foreign Application Priority Data
Jul. 26, 2007 (EP) 07113241.9

(51) Int. Cl. A61N 2/04

(52) U.S. Cl. 600/14

(57) ABSTRACT

Device for applying an electromagnetic field for stimulation of a process within a living organism when applied to at least part of a body, comprising a driver for generating a time varying drive signal and a transducer responsive to said drive signal for generating a time varying electromagnetic field signal B(t). The signal B(t) comprises a superposition of two or more periodic base signals bᵢ(t) (i=1, 2, 3, . . . ). The signal bᵢ(t) is defined as: bᵢ(t)=aᵢ*(2exp(−ωᵢt)−(1+exp(−ωᵢTᵢ/2)) for 0≦t≦Tᵢ/2 and bᵢ(t)=−bᵢ(t−Tᵢ/2) for Tᵢ/2≦t≦Tᵢ, wherein Tᵢ is the period of bᵢ(t), aᵢ is an amplitude of bᵢ(t) and wᵢ is a characteristic frequency determining the shape of the signal bᵢ(t).

### CROSS-REFERENCE TO RELATED APPLICATION

The present application is a national stage filing of International patent application Ser. No. PCT/EP2008/059482, filed Jul. 18, 2008, and published as WO 2009/013249 in English.

### FIELD OF THE INVENTION

Aspects of the invention relate to stimulating a process within living organisms using electromagnetic fields. In particular, an aspect of the invention relates to a device which is suitable for effective stimulation of processes, in particular the immune response, in living organisms.

### BACKGROUND OF THE INVENTION

The discussion below is merely provided for general background information and is not intended to be used as an aid in determining the scope of the claimed subject matter.

It is well known that time varying low-energy electric or magnetic fields produce a range of responses in biological systems. Based on these responses various therapeutic or biostimulation treatments using low frequency and low-energy electromagnetic fields have been proposed. For example, U.S. Pat. No. 3,890,953 describes a method for the stimulation of bone growth and other tissues. In U.S. Pat. No. 5,183,456 a method for regulation of the growth of cancer cells is described. In U.S. Pat. No. 5,290,409 a method is described in which the transport of several types of ions can be influenced.

An interesting group of studies have demonstrated that human and murine macrophages can be stimulated to higher activity through low frequency electromagnetic field exposure (see Simko et al., Eur. J. Cell Biol. Vol. 80, 2001, p. 562-566 and Lupke et al., Free Radic. Res. Vol. 38, 2004, p. 985-993). Several authors have demonstrated that the observed production of cytokines, increased immune parameters and stress effects were initiated by exposure to electromagnetic fields. From these studies it was concluded that low field electromagnetic field exposure causes stress at the cellular level, leading to production of cytokines and consequently biological response, including immune response (see Blank et al., Bioelectrochem. and Bioenerg., Vol. 33, p. 109-114, 1992 and Mol. Biol. Cell Vol. 6, p. 466a, 1995; Goodman et al., Bioelectrochem. and Bioenerg., Vol. 33 p. 115; Simko et al., J. Cell. Biochem. Vol. 93, 2004, p. 83-92; Monselise et al., Biochem. & Biophys. Res. Com. Vol. 302(2), p. 427-434, 2004; De Bruyn et al., Environ. Res., Vol. 65(1) p. 149-160, 1994; Markov et al. in Bioelectromagnetics edited by S. N. Ayrapetryan and M. S. Markov (eds.), Springer 2006, p. 213-225).

Proper stimulation of the immune response leads to improved resistance against infectious diseases and thus positively affects the health of the exposed organism. This insight opens new possibilities for (preventive) treatment in large, dense populations wherein infectious diseases are an increasing problem. Such problems are especially prevalent in populations with genetically uniform organisms such as farmed livestock, chicken, shrimp and fish populations. Infectious diseases can be very damaging to such populations and treatments are very costly.

WO 03/035176 describes a device which is particularly effective in stimulation of the immune system of humans and animals. This device is adapted to the application of time dependent electromagnetic fields to a part of the body of a living organism. The applied signal has a spectrum of frequencies in which some frequencies or frequency areas are more strongly present than others. Such a device can support a therapy in which afflictions involving inflammation and infection can be treated.

The system for electromagnetic stimulation described in WO 03/035176 is a small-scale system only suitable for the treatment of a single organism. Moreover, no indications regarding the shape of the signal are given. Effective electromagnetic stimulation of large populations on a large-scale and the particular type of signals used therein are not addressed in the prior art. For instance, livestock populations are usually kept in large-area buildings or other spaces of large dimensions. Usually stables a and sheds for cows, chickens and pigs or ponds for breeding fish have typical dimensions of at least tens to hundreds of meters. Without special measures, controlled electromagnetic stimulation of such large areas is difficult and would require large amounts of energy. Moreover, when using the device in more remote areas a battery fed system with a solar and/or wind energy supply is necessary. In that case reduction in power consumption is a very important aspect.

Moreover, the buildings where livestock is kept vary in size and construction. The transducers installed in such buildings are tailor made. Consequently, the impedance of these electromagnetic transducers will—to a certain extent—vary from building to building. These variances and deviations in the load of the driving electronics of the electromagnetic transducers will affect the electromagnetic signal produced. This will negatively influence the effectiveness of the stimulation treatment.

### SUMMARY OF THE INVENTION

This Summary and the Abstract herein are provided to introduce a selection of concepts in a simplified form that are further described below in the Detailed Description. This Summary and the Abstract are not intended to identify key features or essential features of the claimed subject matter, nor are they intended to be used as an aid in determining the scope of the claimed subject matter. The claimed subject matter is not limited to implementations that solve any or all disadvantages noted in the background.

An aspect of the invention relates to the observation that signals of specific shape are required to achieve an effective and beneficial stimulation of the processes within a living organism. It involves the recognition that an electromagnetic signal comprising a superposition of at least two periodic electromagnetic signals of a particular shape is especially effective in the stimulation of biological processes, including stimulation of the immune system. As this electromagnetic signal causes an effective stimulation of the processes, including the immune response, in living organisms, small amplitude signals can be used thereby reducing the amount of energy needed to generate these fields. Moreover, the signal can be generated by using relatively simple and cost effective electric components.

One aspect of the invention relates to a device for applying an electromagnetic field adapted to stimulate processes, such as the immune response, within living organisms when coupled to at least part of a body such organism. The device comprises a driver, such as a digital signal generator, for generating a time varying drive signal and one or more transducers, such as specially adapted electromagnetic coil structures, which are responsive to the drive signal of the signal generator. Preferably the transducer is suitable for generating electromagnetic fields over large areas.

The transducer generates a time varying signal B(t) comprising the electromagnetic field which is very effective for stimulating processes within the body. Signal B(t) comprises a superposition of at least two periodic base functions bᵢ(t) (i=1, 2, 3, . . . ), wherein the functions bᵢ(t) are defined as:

bᵢ(t)=aᵢ*(2exp(−ωᵢt)−(1+exp(−ωᵢTᵢ/2)) for 0≦t≦Tᵢ/2

bᵢ(t)=−bᵢ(t−Tᵢ/2) for Tᵢ/2≦t≦Tᵢ

Tᵢ is the period of the function bᵢ(t), aᵢ is an amplitude of the function and ωᵢ is a characteristic frequency which determines to a large extent the shape of the signal bᵢ(t). It has been experimentally determined that such an electromagnetic field provides an effective stimulation of a process within the body of a living organism.

Typically, the amplitude aᵢ (i=1, 2, 3, . . . ) is chosen such that the peak amplitude of B(t) at the treatment positions will be in the range between 1 nT to 1 mT, preferably within the range between 0.03 μT to 30 μT. Due to the effective shape of signal B(t) for electromagnetic stimulation, even small amplitudes signals will be sufficient to generate advantageous stimulation. The use of this signal thus drastically reduces energy consumption in large area and large scale applications.

In one embodiment each of said characteristic frequencies ωᵢ (i=1, 2, 3, . . . ) is chosen to substantially match a desired characteristic frequency ωₒ. This way all base functions bᵢ(t) have the same characteristic frequency ωₒ. Typically, the characteristic frequencies ωᵢ or the common characteristic frequency ωₒ are chosen from a range between 200 and 20,000 rad.s⁻¹, more preferably between 500 and 15,000 rad.s⁻¹, in particular between 1000 and 5,000 rad.s⁻¹.

In one aspect of the invention the characteristic frequency ω₁ of the transducer, which is determined by the R/L ratio, is chosen to substantially match the desired characteristic frequency ωₒ of the signal. Here, R represents the resistance and L the inductance of the inductive coil(s) in said transducer. If the transducer is driven by a block-wave type drive signal an electromagnetic signal B(t) is generated which has optimal stimulation effects. Particular transducer structures are described in more detail in a related application with title “Coil structure for electromagnetic stimulation of processes within a living organism, device using such coil structure and method of driving”, which is hereby incorporated by reference in this application.

In an embodiment of the invention at least one of the periods Tᵢ (i=1, 2, 3 . . . ) is chosen from a range between 0.01 ms and 1000 ms, preferably between 0.1 ms and 100 ms. Typically periods Tᵢ (i=1, 2, 3 . . . ) have different values. Preferably, at least one of said periods Tᵢ substantially matches one of a first group of periods Tᵢ′=1/fᵢ or a second group of periods Tᵢ″=(B_{loc}/Bₒ)·(1/ fᵢ) wherein f₁=10 Hz, f₂=700 Hz, f₃=750 Hz, f₄=2200 Hz. B_{loc} is the local earth magnetic field at the position of the device and Bₒ=47 μT. Here, the scaling behavior of the frequency with the B_{loc}/Bₒ-ratio was experimentally determined. Scaling behavior with the ambient magnetic field was also observed in U.S. Pat. No. 5,290,409.

Selection of the periods Tᵢ (i=1, 2, 3 . . . ) according to one or a combination of the above mentioned selection criteria will provide an electromagnetic field signal which is particularly effective and advantageous for use in electromagnetic stimulation in large-scale and large-area applications.

In an embodiment of the invention the device comprises a signal generator which supplies a signal to an amplifier for driving the electromagnetic transducer. Commonly known linear amplifiers are not suitable for driving large area transducers. Such an amplifier would consume too much energy. In one embodiment of the invention the amplifier is a switching amplifier, for example, a pulse width modulation amplifier or a class D amplifier. Such amplifiers have a high power conversion efficiency and reduced power dissipation. As a result less cooling is needed thereby allowing compact and simple circuitry.

In yet a further aspect of the invention the driver is adapted to generate block-wave signals, preferably adapted to produce a driving voltage signal V(t) comprising one block-wave signal or, preferably, a superposition of at least two block-wave signals vᵢ(t)(i=1, 2, 3, . . . ) wherein each of the block-wave signals vᵢ(t) has a corresponding period Tᵢ. Block-wave signals can be easily generated by a digital signal generator and make optimal use of the voltage power supply in the device. In one embodiment the device is battery fed.

In a further embodiment the device comprises a signal compensator for compensating the drive signal for variations in the characteristic frequency ω₁=R/L of the transducer. Such variations originate from variations in the impedance of the transducer.

The compensator can be arranged between the signal generator and the amplifier. The compensator comprises an active circuit with a characteristic frequency which substantially matches the desired characteristic frequency ωₒ of the signal.

In one embodiment the compensator comprises an RC circuit wherein the resistor Rₒ and the capacitor Cₒ of the RC circuit is chosen such that the RₒCₒ product substantially matches the desired characteristic frequency ωₒ of the signal. The use of the RC circuit thus allows very simple and cost effective load adjustments and eliminates and/or reduces the detrimental effects of variations in the impedance of the transducer on the desired shape of the electromagnetic field signal.

The compensator can also comprise an inductive active circuit or a combined capacitive/inductive active circuit having at least one characteristic frequency which matches the desired characteristic frequency ωₒ of the signal. The compensator thus allows the device to generate an electromagnetic signal of a preferred shape regardless of variations in the impedance of the transducer.

The invention further relates to a device for electromagnetic field stimulation of a process within a living organism when applied to at least part of a body, which comprises a driver for generating a time varying drive signal, at least one transducer responsive to said drive signal for generating a time varying electromagnetic field and wherein said electromagnetic field contains a superposition of at least two periodic functions, each of said functions having a characteristic frequency ωₒ determining the shape of said functions. The device further comprises a pulse width modulation amplifier and/or signal compensator for compensating said drive signal for deviations in the characteristic frequency of said transducer ω₁=R/L from said characteristic frequency ωₒ arranged between said driver and said transducer. The use of a pulse width modulation amplifier and/or the signal compensator in the device provides very effective driving electronics for large area and large scale electromagnetic transducers for stimulation of a process within a living organism.

### BRIEF DESCRIPTION OF THE DRAWINGS

Aspects of the invention will be further explained by means of the description of exemplary embodiments, reference being made to the following figures:

FIG. 1 represents a schematic drawing of a device according to an embodiment of the invention.

FIG. 2 represents a schematic drawing of the shape of a preferred periodic base signal bᵢ(t).

FIG. 3 illustrates the results of experiments on phagocyte cells treated with an electromagnetic field signal.

FIG. 4 illustrates the results of in vivo experiments on infected fantail goldfish treated with an electromagnetic field signal.

FIG. 5 illustrates the results of in vivo experiments on infected chicken broilers treated with an electromagnetic field signal.

FIG. 6 represents a graph regarding the improved feed conversion of chicken broilers treated with an electromagnetic field signal.

FIG. 7 represents a schematic drawing of the driving electrons of a driver according to an embodiment of the invention.

### DESCRIPTION OF EXEMPLARY EMBODIMENTS

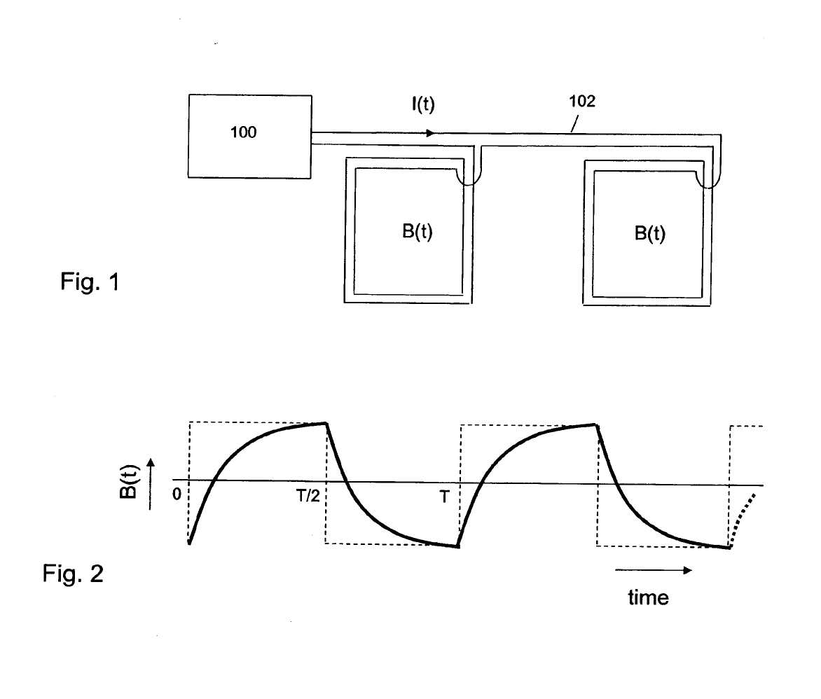

FIG. 1 shows a schematic representation of a device for electromagnetic stimulation. The device comprises a driver 100 for generating a voltage signal V(t) which drives the electromagnetic transducer 102. The transducer 102 comprises one or more electromagnetic coils having together a certain inductance L and resistance R. In response to the driving signal V(t), a current I(t) runs through the transducer, generating an electromagnetic field B(t). Typically, in large area and large scale applications the electromagnetic coils form distributed coil structures. These distributed structures are arranged over or under a surface area S on which the living organisms are kept. The specific transducer structures are described in more detail in a related application with title “Coil structure for electromagnetic stimulation of processes within a living organism, device using such coil structure and method of driving”.

The driver generates a driving signal which is fed to the electromagnetic transducer. In response, the transducer generates a time varying signal B(t) comprising an electromagnetic field which is very effective in stimulating processes within the body. The low frequency electromagnetic signal B(t) comprises a single base signal or, preferably, a composite signal. The composite signal contains a superposition of at least two periodic base signals bᵢ(t) (i=1, 2, 3, . . . ) wherein each of these base signals has a shape as illustrated in FIG. 2. The periodic base signal bᵢ(t) is defined as:

bᵢ(t)=a*(2exp(−ωᵢt)−(1+exp(−ωᵢTᵢ/2)) for 0≦t≦Tᵢ/2

bᵢ(t)=−bᵢ(t−Tᵢ/2) for Tᵢ/2≦t≦Tᵢ

Here Tᵢ is the period of the signal bᵢ(t), aᵢ is an amplitude of the signal and ωᵢ is a characteristic frequency of the signal. The characteristic frequency ωᵢ determines the rise and fall time of the signal and thus determines to a large extent the shape of the signal. The superposition of the signals includes a summation or an integration of two or more base signals, preferably having different frequencies. When applying such time varying signal to a part of the body, the different ions involved in the biochemical processes in the cells are subjected to an electromotive force which is proportional to the time derivative of time varying magnetic field dB(t)/dt. Hence, the forces on the ions in the cells can be manipulated by tuning the characteristic frequency of the base signals. The applicant found that the use of two or more base signals having a particular shape determined by the characteristic frequency provides surprisingly effective stimulation of the physiological processes in the cells.

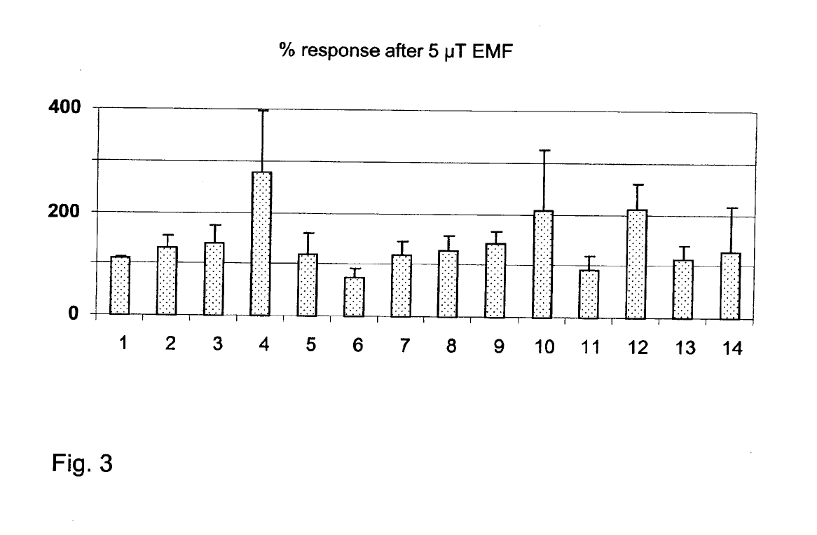

The graphs in FIGS. 3 to 6 show results from in vitro and in vivo experiments in which effects on the immune response were explored to various pathogens of exposure using the composite low frequency electromagnetic signal of the present invention. The signal comprised shaped waveforms bᵢ(t) as described in relation to the base signals of FIG. 2. Typically, base frequencies fᵢ=1/T, between 250 and 5000 Hz were used. The experiments described in the FIGS. 3 to 6 relate to a daily, 30 minutes electromagnetic stimulation treatment using a signal composed of the base frequencies 700 and 750 Hz. The functions bᵢ(t) were chosen to have the same characteristic frequency ωₒ of around 1900 rad.s⁻¹. Various electromagnetic field strengths between 100 nT and 50 μT were used.

FIG. 3 shows the results of a series of in vitro experiments on phagocytes. The figure depicts the Oxygen burst in phagocyting cells relative to the control wherein each run represents 48 samples (total confidence level p<0.0001). Reactive oxygen species (ROS) production in electromagnetically stimulated common carp head kidney-derived phagocytes was determined as a measure for immune activation. The measurements were based on the reduction of the salt nitro blue tetrazolium (NBT) by oxygen. Such reduction results in a blue coloration and can be measured using spectrophotometrics. From the experimental results it followed that exposure to an electromagnetic field of 5 μT and 1.5 mT led to 42% and 33% increase in immune activity, respectively, compared to negative control values.

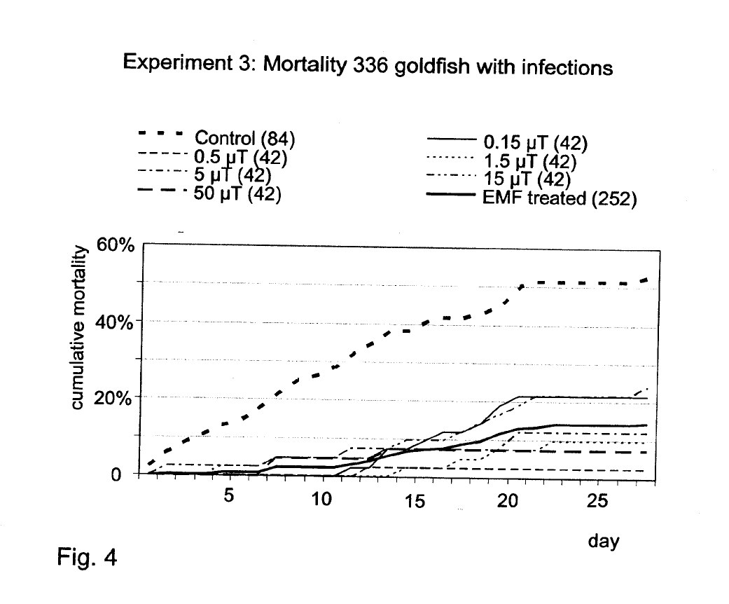

FIG. 4 shows results in vivo experiments on fantail goldfish (Carrassius Auratus spp.). Electromagnetic stimulation experiments were performed using six different field strengths ranging from 0.15 μT to 50 μT. The goldfish were heavily infected with Ecto parasites (Gill parasites) such as Dactylogyrus/Gyrodactylus, Trichodina, Chilodinella and Costia. These types of parasite infections occur frequently at the breeding stage of the fish and increase in intensity during storage and international transport due to the fact that large populations are packaged in one volume. Such infections and subsequent secondary bacterial infections cause high mortality if not treated.

The results in FIG. 4 show that the control group suffered a mortality rate up to 52% on day 28. In contrast, the average mortality rate of the electromagnetically treated fish was 15% at day 28. The effectiveness of the treatment reduces when using fields smaller than 0.05 μT. These results were reproducible and show that the low energy electromagnetic treatment using the composite electromagnetic signals generated by the device of the present invention results in a decrease in mortality at all field strength levels used.

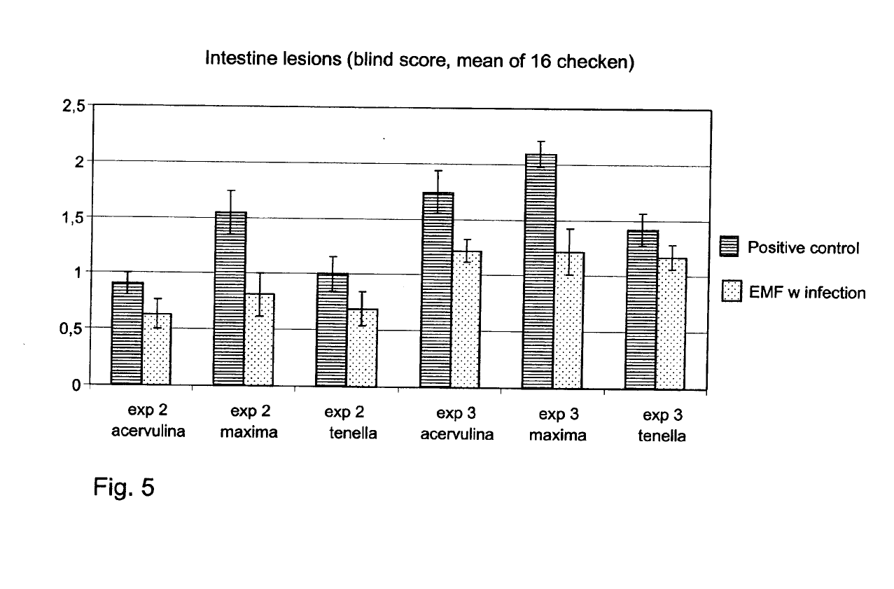

FIG. 5 illustrates a series of in vivo experiments on 560 commercial broiler chickens, which were exposed to infection pressure from Coccidiosis. The graphs show that Coccidial lesion of intestines due to Eimeria Acervulina and Eimeria Maxima were significantly lower in group treated with an electromagnetic field. Treatment with a 6.5 μT composite electromagnetic field signal reduced intestinal lesions up to 40%.

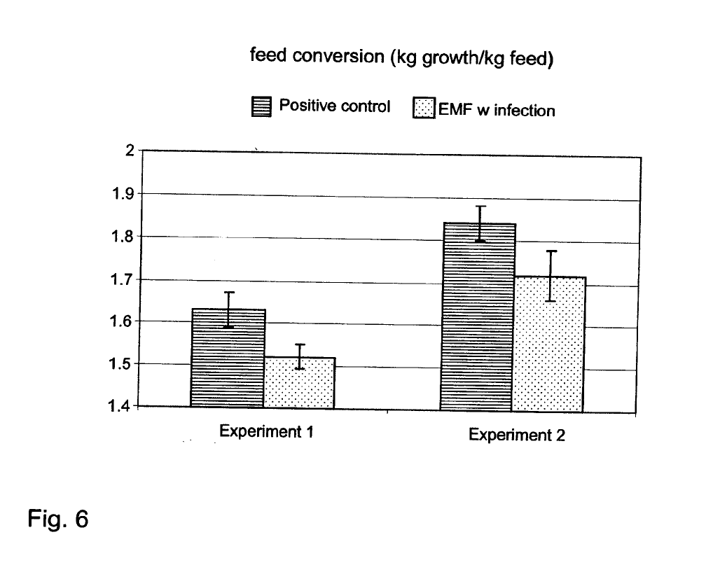

FIG. 6 depicts the feed conversion (i.e. the ratio between the growth of the chickens in kilograms and the feed in kilograms) of treated and non-treated chickens in the experiments as described in relation to FIG. 5. A significant and economically relevant improvement in feed conversion up to 8% is achieved by electromagnetic treatment of chickens with Coccidiosis infection. The improvement indicates that the electromagnetic stimulate the health and thus the growth per unit of feed of the chickens.

Further experiments show that particular effective stimulation can be achieved by selecting base frequencies from a first group of frequencies f₁=10 Hz, f₂=700 Hz, f₃=750 Hz, f₄=2200 Hz and/or a second group of frequencies equal to the frequencies of the first group multiplied with a factor B_{loc}/Bₒ, wherein B_{loc} is the local earth magnetic field at the position of the device and Bₒ=47 μT.

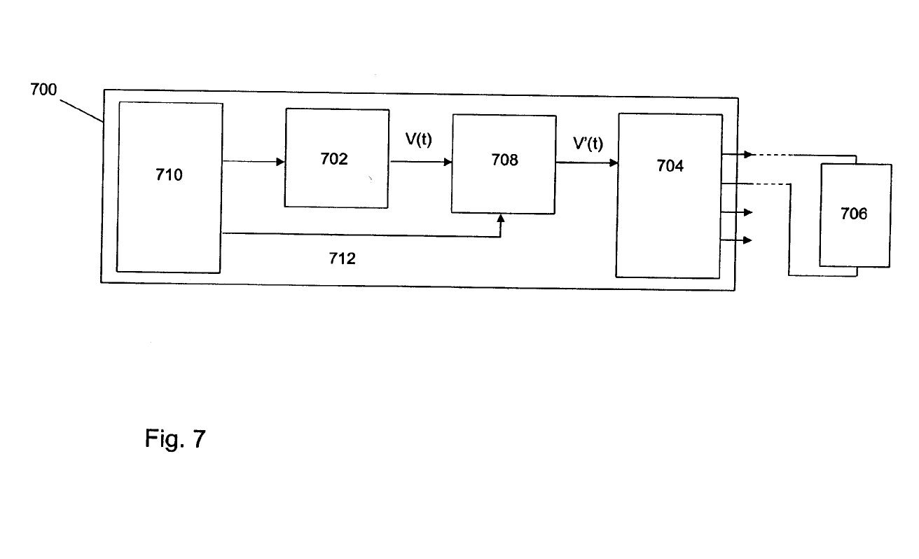

The electromagnetic signal is generated by a driver 700 comprising driving electronics as schematically illustrated in FIG. 7. A signal generator 702 provides a driving signal V(t) to the input of one or more amplifiers 704. The signal generator 702 is typically a digital signal generator, which is capable of generating a driving signal V(t) comprising one block-wave signal or, preferably, a superposition of at least two block-wave signals vᵢ(t)(i=1, 2, 3, . . . ), wherein each of the block-wave signals vᵢ(t) has a corresponding period Tᵢ. Preferably, the base functions bᵢ(t) have the same characteristic frequency ωₒ. In that case the desired shape of the signal is determined by choosing the characteristic frequency ω₁=R/L of the inductive coil(s) in the transducer 706 to match approximately the desired characteristic frequency ωₒ.

The driver further comprises a compensator 708 which is arranged between the signal generator 702 and the amplifiers 704 as shown in FIG. 7. The compensator 708 is able to compensate for deviations Δω between the desired characteristic frequency ωₒ and the characteristic frequency ω₁=R/L of the inductive coil(s). These deviations Δω are caused by various reasons such as geometrical variations in impedance of the coils or (geometrical) restraints in matching ω₁ to the desired characteristic frequency ωₒ.

In order to generate the desired electromagnetic field B(t), a current I(t) should be generated in the coil(s) of the transducer 706. This is done by applying a voltage signal V(t) comprising one or, preferably, a superposition of at least two block-wave signals vᵢ(t)(i=1, 2, 3, . . . ) to the input of one or more amplifiers which drive the transducer. Here the characteristic frequency of the transducer ω₁=R/L approximately matches the characteristic frequency ωₒ of the desired signal. If however ω₁ deviates with a value Δω from ωₒ then an adjusted voltage V′(t)=V(t)−LΔωI(t) should be generated in order to obtain the desired electromagnetic field signal B(t). V′(t) could be generated digitally, however this solution requires expensive signal processing hardware.

In one aspect of the invention a compensator 708 allows the generation of the adjusted voltage V′(t) with simple low power, analog components so that deviations in the impedance of the transducer are compensated. In the compensator 708 the voltage V(t) of the signal generator is applied to an RC circuit having a resistor Rₒ and a capacitor Cₒ such that the RₒCₒ product substantially matches the desired characteristic frequency ωₒ of the signal. Here a relatively high resistance Rₒ can be chosen such that the energy dissipation in the circuit can be kept low. By using simple analog addition and subtraction circuitry, which is well known in the art, the adjusted voltage V′(t) can be constructed in a simple way even when V(t) is a more complex signal constructed by the addition of various block-wave functions vᵢ(t).

The use of the RC circuit thus allows very simple and cost effective load adjustments and eliminates and/or reduces the detrimental effects of variations in the impedance of the transducer on the desired shape of the electromagnetic field signal. The compensator can also comprise inductive active circuitry or combined capacitive/inductive active circuitry having at least one characteristic frequency which substantially matches the desired characteristic frequency ωₒ of the signal.

The voltage V(t) of the signal generator or, when applicable, the compensated voltage signal V′(t) is preferably offered to the input of a pulse width modulation amplifier or a class D amplifier, which have a high power conversion efficiency and reduced power dissipation compared to a conventional linear amplifier. As a result less cooling is needed when thereby allowing compact and simple circuitry. The energy considerations in the design of the driver are especially important when the driver is battery fed, which is required when the stimulation treatment is used in more remote areas.

The driver in FIG. 7 can further comprise a processor 710 for control and automation of the signal generation processes. For instance the driver can include further circuitry which is able to determine the characteristic frequency ω₁ of the transducer. Using this frequency the processor can instruct the compensator via a control line 712 to generate a compensated voltage signal V′(t).

The invention is not limited to the embodiments described above, which may be varied within the scope of the accompanying claims.

1. A device for applying an electromagnetic field for stimulation of a process within a living organism when applied to at least part of a body, comprising:
a driver configured to generate a time varying drive signal,
at least one transducer responsive to said drive signal configured to generate a time varying signal B(t) comprising said electromagnetic field, and
wherein said signal B(t) comprises a superposition of two or more periodic signals bᵢ(t) (i=1, 2, 3, . . . ), each signal bᵢ(t) being defined as:
bᵢ(t)=aᵢ*(2exp(−ωᵢt)−(1+exp(−ωᵢTᵢ/2)) for 0≦t≦Tᵢ/2
bᵢ(t)=−bᵢ(t−Tᵢ/2) for Tᵢ/2≦t≦Tᵢ
wherein Tᵢ is the period of bᵢ(t), aᵢ is an amplitude of bᵢ(t) and ωᵢ is a characteristic frequency determining the temporal shape of the signal bᵢ(t).

2. The device according to claim 1, wherein said characteristic frequencies ωᵢ (i=1, 2, 3, . . . ) are chosen from a range between 200 and 20,000 rad.s⁻¹, more preferably between 500 and 15,000 rad.s⁻¹, in particular between 1000 and 5,000 rad.s⁻¹.

3. The device according to claim 1, wherein at least one of said periods Tᵢ (i=1, 2, 3 . . . ) is chosen from a range between 0.01 ms and 1000 ms, preferably between 0.1 ms and 100 ms.

4. The device according to claim 1, wherein at least two of said periods Tᵢ (i=1, 2, 3 . . . ) are chosen to have different values.

5. The device according to claim 1, wherein at least one of said periods Tᵢ substantially matches one of the periods defined by a first group of periods Tᵢ′=1/fᵢ or a second group of periods Tᵢ″=(B_{loc}/Bₒ)·(1/fᵢ) wherein f₁=10 Hz, f₂=700 Hz, f₃=750 Hz, f₄=2200 Hz, B_{loc}, is the local earth magnetic field at the position of the device and Bₒ=47 μT.

6. The device according to claim 1, wherein said device further comprises an amplifier arranged between said driver and said transducer.

7. The device according to claim 1, wherein said driver comprises a signal generator adapted to generate a driving signal V(t) comprising one block-wave signal or a superposition of at least two block-wave signals vᵢ(t)(i=1, 2, 3, . . . ), wherein each of said block-wave signals vᵢ(t) has a corresponding period Tᵢ.

8. The device according to claim 1, wherein each of said characteristic frequencies ωᵢ (i=1, 2, 3, . . . ) substantially matches the characteristic frequency of said inductive coil ω₁=R/L.

9. The device according to claim 1, wherein each of said characteristic frequencies ωᵢ (i=1, 2, 3, . . . ) substantially matches a characteristic frequency ωₒ and wherein said device further comprises a signal compensator configured to compensate said drive signal for deviations in the characteristic frequency of said transducer ω₁=R/L from said characteristic frequency ωₒ.

10. The device according to claim 9, wherein said signal compensator is arranged between said driver and said amplifier.

11. The device according to claim 9, wherein said signal compensator comprises an RC circuit wherein resistor Rₒ and capacitor Cₒ of said RC circuit is chosen such that the product Rₒ·Cₒ substantially matches said characteristic frequency ωₒ.

12. A device for electromagnetic field stimulation of a process within a living organism when applied to at least part of a body, comprising:
driver configured to generate a time varying drive signal,
at least one transducer responsive to said drive signal configured to generate a time varying electromagnetic field and wherein said electromagnetic field contains a superposition of at least two periodic functions, each of said functions having a characteristic frequency ωₒ determining the shape of said functions,
wherein said device further comprises a pulse width modulation amplifier and/or a signal compensator configured to compensate said drive signal for deviations in the characteristic frequency of said transducer ωᵢ=R/L from said characteristic frequency ωₒ arranged between said driver and said transducer.

13. A method for applying an electromagnetic field for stimulation of a process within a living organism when applied to at least part of a body, comprising:
generating a time varying drive signal,
using at least one transducer responsive to said drive signal for generating a time varying signal B(t) comprising said electromagnetic field, and
wherein said signal B(t) comprises a superposition of at least two periodic signals bᵢ(t) (i=1, 2, 3, . . . ), each signal bᵢ(t) being defined as:
bᵢ(t)=aᵢ*(2exp(−ωᵢt)−(1+exp(−ωᵢTᵢ/2)) for 0≦t≦Tᵢ/2
bᵢ(t)=−bᵢ(t−Tᵢ/2) for Tᵢ/2≦t≦Tᵢ
wherein Tᵢ is the period of bᵢ(t), a, is an amplitude of bᵢ(t) and ωᵢ is a characteristic frequency determining the shape of the signal bᵢ(t).

14. The method of driving a transducer in a device according claim 13, wherein said transducer is driven by a driving signal V(t) containing a superposition of two or more block-wave signals vᵢ(t)(i=1, 2, 3, . . . ), wherein each of said block-wave signals vᵢ(t) has a corresponding period Tᵢ.
